(19) United States
(12) Patent Application Publication
(10) Pub. No.: US 2008/0264426 A1
(43) Pub. Date: Oct. 30, 2008

(54) DEVICE TO TREAT SNORING AND OBSTRUCTIVE SLEEP APNEA IN ADULTS AND TO PREVENT INFANTS FROM SLEEPING NON-SUPINE

(76) Inventor: James Walker, Farmington, UT (US)

Correspondence Address: James Walker, 1726 Hampton CT, Farmington, UT, 84025, US

(21) Appl. No.: 12/111,134

(22) Filed: Apr. 28, 2008

Related U.S. Application Data
(60) Provisional application No. 60/914,754, filed on Apr. 29, 2007.

(51) Int. Cl. A61F 5/56; A61B 5/11

(52) U.S. Cl. 128/848; 600/300

(57) ABSTRACT

This is a position sensing monitor that provides private sensory biofeedback to adults, in the form of auditory stimulus or tactile sensation (vibration), to promote non-supine sleep. The essential feature of this invention is to eliminate/reduce position-related snoring and obstructive sleep apnea and not disrupting sleep of bed partner. A variation of this concept is its use in infants to promote sleeping supine by parental notification of rolling from supine to side or prone positions, allow re-position to supine and thereby reducing the risk for SIDS.

This application is a claims benefit of my provisional application No. 60/914,754 filed 29-APR-2007.

### FIELD OF THE INVENTION

The present invention is a small supine/non-supine sleeping position monitor, worn on the chest and based upon biofeedback principles, to treat snoring and obstructive sleep apnea in adults without disturbing bed-partner's sleep. An auditory and/or tactile stimulus is presented whenever an individual is in the supine position. A variation of this monitor is used to prevent sleeping on sides or in the prone position in infants by alerting nearby parents that there has been a change in the infant's body position. Accordingly, this invention involves the fields of electronics, medicine, and other health sciences.

### BACKGROUND OF THE INVENTION

In the past two decades, it has become recognized that sleep disorders are pervasive; approximately 40 million Americans suffer from snoring and 20 million have sleep apnea. Snoring occurs when there is an partial obstruction of the upper airway caused by enlarged tissues (such as the tonsils or uvula), if structures surrounding the passageways thicken (as with weight gain), or there is closure of the airway due to the effects of gravity (as sleeping supine). Most of the anatomical and physiological correlates of snoring appear to be due to the effect of gravity on the upper airway. Obstructive sleep hypopnea is further but not complete closure of the upper airway whereas obstructive sleep apnea is a complete closure of the airway; both lasting at least ten seconds. A noted sleep physician and researcher, William Dement, M.D., Ph.D., co-director of the Stanford Sleep Research Center and professor of psychiatry and behavioral sciences at Stanford University has stated “Upper airway obstructions can destroy the restorative qualities of sleep and often severely affect the health of a vast number of people in the United States and around the world.” He stated further said “There is a pressing need in the medical community and healthcare industry to develop new, minimally invasive therapies for these serious health conditions. Technological innovations can bring more tolerable and less painful treatments to millions of sufferers and help them breathe, sleep and live more healthily.”

Snoring has been determined to be a risk factor for ischemic heart disease, hypertension and stroke. This is due, in part, because of the association of snoring with sleep apnea. Sleep apnea has now been determined to be an independent risk factor for hypertension, heart disease, stroke, and diabetes; appropriate treatment can reduce the risks for these conditions. In 1998, National Commission on Sleep Disorders Research stated that sleep apnea results in approximately 38,000 cardiovascular deaths annually in the United States. However, given the recent findings of sleep apnea as a risk factor for coronary artery disease; the link between sleep apneas as a causative factor for hypertension and left ventricular hypertrophy; and strong association between sleep apnea and stroke, this figure is likely a gross underestimated of deaths due to sleep apnea. That is, more the overall mortality from sleep apnea likely exceeds that of breast cancer.

The aggravating effect of the supine body position on breathing abnormalities during sleep was recognized from the earliest studies on sleep breathing disorders; both in terms of snoring and obstructive sleep apnea. It has been shown in a large proportion of obstructive sleep apnea patients have positional sleep apnea; i.e., there at least twice as many apneas/hypopneas during sleep in the supine posture as in the lateral position. Sleeping supine not only increases the frequency of the abnormal breathing events but also their severity in terms of length and associated declines in arterial oxygen saturation.

With the acknowledgement of the importance of correcting sleep apnea, there has been the development of new discipline of sleep medicine. This field has recently been recognized by the American Board of Medical Specialties, the pre-eminent entity development of practice standards and physician overseeing certification in the United States. In a 2006 position statement, the Standards of Practice Committee of the American Academy of Sleep Medicine in “Practice Parameters for the Medical Therapy of Obstructive Apnea” recommended standardized approaches for the maintenance of non-supine position during sleep. Possible means to maintain non-supine sleep include positional alarms, sleep shirts, and special pillows. Although the most promising concept is that of a positional alarm, it suffers the potential drawback of not only awakening an individual with snoring or apneas, but also awakening the bed partner.

In infants, a converse of the situation is the rule; i.e., infants should sleep on their backs and avoid sleeping on their sides or prone in order to reduce the risk for sudden infant death syndrome (SIDS). Since 1992, the American Academy of Pediatrics has recommended that infants be placed on their backs to reduce the risk for SIDS. This was reaffirmed in 2000 but was also amended to include the recommendation that infants not sleep on their sides. Although SIDS generally occurs between the ages of 2-4 months, it can occur up to one year in age. The milestones published by the National Institute of Child and Human Development for infants rolling over are:
Infants 1-4 months: usually do not have the ability to roll over.
Infants 5-9 months: are learning to master rolling from back to side.
Infants 10 months and older: have mastered ability to roll from back to stomach.

However, some infants may begin to rollover earlier or later than indicated above.

The prevalence of US SIDS rates has dramatically decreased since the American Academy of Pediatrics adopted its “Back to Sleep program” in 1994 affirming the importance of sleeping supine. The present invention is a means to more accurately monitor infant sleeping position.

### DETAILED DESCRIPTION

The present invention provides a method to monitor sleeping supine and to generate an alerting signal when this condition is met (adults) or not met (infants).

A. Treatment of Snoring and Obstructive Sleep Apnea in Adults. This monitor is a private alerting system that would be used to eliminate/reduce snoring or position-related apneas/hypopneas. This monitor differs from previous systems in that it allows notification of supine sleeping events without disturbing bed partner through the use of tactile sensation or a personal auditory signal presented to the individual using it.

Snoring In Adults. As outlined in the BACKGROUND OF THE INVENTION, snoring is positional-related, occurring in most frequently and with greatest intensity when sleeping supine. Most couples sleeping together have either been the recipient of being requested to move from their back to the lateral position to sleep in order to eliminate/reduce snoring or the instigator of such a request. Often the butt of jokes, snoring in its worst form, can indicate underlying medical problems or at the minimum, cause socio-marital problems. There are a wide variety of non-prescription treatments with limited scientific evidence of efficacy. However, there is clear scientific documentation that re-positioning from supine to other sleeping positions markedly reduces or eliminates snoring. The monitor would be put on at bedtime and activated once a person is ready to sleep. It would be held in place by a thoracic band, an adhesive, a small holster attached to bed clothing, or Velcro attached to bed clothing. When shifting to the supine position for more than 30-seconds, a personal alarm is generated and continues intermittently until there is a shift to a non-supine position. The alerting signal is a pulsating tone of varying intensity (<85 cB), a vibrating tactile sensation, or a combination of both. In order not to awaken the bed partner, the tone is transmitted to a small bud type personal earphone, either by wire or wirelessly. Once the sleeping individual re-positions, the stimuli (tone or vibration, or both) ceases. The basis for determining position changes is an accelerometer, which can also measure with great sensitivity, activity. If activity is not recorded for an hour period, the monitor shuts off, thereby conserving its battery.

Obstructive Sleep Apnea In Adults. The principles outlined above for snoring also apply to reducing position-related apneas/hypopneas. During the diagnosis of sleep apnea with a polysomnogram (multi-channel bio-signal sleep study), standard parameters reported are the supine sleep apneas/hypopnea index (number of events/hr) compared to non-supine sleep apneas/hypopnea index. Consequently, the effects of position therapy can be determined prior to the implementation thereof.

One aspect of the present invention is the capability to store body position information over a period of time for the elimination/reduction of either snoring or obstructive sleep apnea. Consequently, it can be used as a trainer to condition an individual to sleep non-supine. Once data polling indicates that the individual is not sleeping supine, then it would only be used periodically to assess compliance to sleeping non-supine.

Another embodiment of the monitor is to access the adequacy of other methods of preventing supine sleep. For an example, The U.S. Food and Drug Administration have approved a pillow monitor for snoring and mild sleep apnea; it is meant to position the neck so the airway is more likely to remain open. However, with the exception of spouse observations, there is no means to assess whether a person sleeps supine or not.

B. Alert System for Maintain Infants in the Supine Position. A variation of this monitor is a system to alert parents when infants roll over from their back (preferred position) to either their sides or prone. As noted, earlier, the preferred body position in infants is supine with non-supine positions (side or prone) associated with increased risks for SIDS. In the embodiment of the present system, an alerting signal be emitted when an infant moves to a non-supine position. The alerting signal could be an audible alarm at crib side or one that is transmitted wirelessly to nearby parents informing them that infant has repositioned to a non-supine position, which in turn would allows parents to place the infant in a supine position.

What is claimed is:

1. A small monitor that identifies supine and non-supine sleeping position.

2. A system as in claim 1 to treat snoring and obstructive sleep apnea in adults without disturbing bed-partner's sleep

3. A system as in claim 2, that is based upon biofeedback principles and provides an intermittent audible tone and/or tactile stimulation when an adult individual is supine position, and ceasing once move to a non-supine position.

4. A system as in claim 3 where the audible can be provided privately by means of a small bud earphone, either wireless or connected by fine wire to the monitor, to not to disturb the individual's bed partner.

5. A system as in claim 2 is worn on chest by means of a strap, adhesive material that attaches to the skin, or attaches to bed clothing

6. A system as in claim 2 that, by learning principles, can condition an individual from sleeping supine.

7. A system as in claim 2 that can store information regarding time spent in the supine and non-supine position.

8. A system as in claim 1 where is attached to an infants bed clothing.

9. A system as in claim 8 that emits an audible alarm to notify parents when an infant moves to a non-supine position.
